(19) United States
(12) Patent Application Publication
(10) Pub. No.: US 2001/0044394 A1
(43) Pub. Date: Nov. 22, 2001

(54) Dual layer transparent and synthetic detergent toilet bar

(76) Inventor: Marty van der Hagen, Leander, TX (US)

Correspondence Address: Rick B. Yeager, 10805 Mellow Lane, Austin, TX, 78759, US

(21) Appl. No.: 09/851,034

(22) Filed: May 7, 2001

Related U.S. Application Data
(63) Continuation-in-part of application No. 09/536,025, filed on Mar. 27, 2000, now abandoned.

(51) Int. Cl. A61K 7/50

(52) U.S. Cl. 510/141; 510/146; 510/147; 510/153; 510/155

(57) ABSTRACT

A transparent toilet bar with a dual-layer pattern of glycerin-based colored transparent soap formulations, and syndet-based soap compositions; and a method for producing the bar. The preferred embodiment of the bar is a dual-layer pattern with a glycerin layer on top and a syndet layer on the bottom. The syndet layer includes 8-12% by weight of water.

### RELATED APPLICATIONS

This application is a continuation-in-part of application Ser. No. 09/536,025 which is abandoned.

### FIELD OF INVENTION

The present invention relates to a decorative article of soap comprised of a glycerin based-soap and synthetic detergent.

### BACKGROUND

The present invention relates to a process for the preparation of soap articles containing a decorative dual-phase pattern in which one layer is a glycerin based-soap, and the second layer is a solid synthetic detergent (“syndet”) formulation.

Transparent soaps are popular with consumers, and many consumers desire decorative soap articles comprised of transparent soap formulations of relatively gentle character.

The prior art includes numerous formulations and process for transparent and translucent soaps. U.S. Pat. No 4,584,126 issued to Joshi on Apr. 22, 1986 provides a background on formulations and manufacturing processes for translucent and transparent soaps.

The prior art also includes transparent soaps with embedded soap shapes, printed films, and other articles. U.S. Pat. No. 5,217,639 issued Jun. 8, 1993 to Mottola describes a dual-phase toilet bar having a first portion that is at least translucent and a second portion that is opaque where the first and second portions are 80-90+% identical in formulation.

The prior art includes various formulations for “combo bars” where both soap and synthetic detergent are combined to a single composition. U.S. Pat. No. 4,851,147 issued Jul. 25, 1989 to Esposito et al describes a transparent soap formulation containing soap and a water soluble synthetic detergent. The bar comprises a fatty acid soap, a polyol, a sugar, and a triethanolamine. U.S. Pat. No. 5,154,849 issued Oct. 13, 1992 to Visscher et al describes a transparent soap bar composition comprising a fatty acid soap, a synthetic surfactant, and moisturizers. U.S. Pat. No. 4,963,284 issued Oct. 16, 1990 to Novakovic et al describes a translucent combo bar with a composition comprising fatty acid soaps, a synthetic organic detergent, polyols, and a triethanolamine. The current invention presents the soap as a distinct transparent glycerin formulation component and the synthetic detergent as distinct opaque component so that the resulting bar is both functional and pleasing in appearance.

The present invention provides a unique and aesthetically pleasing transparent toilet bar incorporating a decorative dual-phase colored pattern.

### SUMMARY OF THE INVENTION

The current invention is a toilet bar comprising a decorative dual-layer pattern comprised of a transparent soap and a syndet soap. An important inventive element is the unexpected result that relatively large amounts of water can be successfully added to the syndet formulation in order to provide a good bond to the transparent soap layer.

The preferred embodiment of the bar is a dual-phase circular bar with a glycerin top layer, and a syndet bottom layer. Coloring and glittering agents may be included in either layer.

The preferred method of production is first to prepare the glycerin soap composition and the syndet soap composition. At this stage, color or glitter may be added. The glycerin composition is poured into a mold until the mold cavity is approximately half full. The glycerin layer is then allowed to cool. After cooling, the syndet composition is poured over the glycerin layer, until the mold is full. The entire soap article is then allowed to cool, at which time it is removed from the mold and packaged.

### BRIEF DESCRIPTION OF THE DRAWINGS

The above-mentioned and other objects and features of the present invention will become apparent from a reading of the following detailed description with reference to the accompanying drawings, in which:

FIG. 1 is a top view of a finished bar showing a dual-layer soap article.

FIG. 2 is a cross section of a finished bar showing a dual-layer soap.

### DETAILED DESCRIPTION OF EMBODIMENT

The term “transparent” as used in this specification is intended to connote its usual dictionary definition. A transparent soap allows ready viewing of objects behind it; while a translucent soap will allow light to pass through in a scattered manner that makes difficult to clearly identify objects behind the translucent soap. A practical test for transparency is to place a ¼ inch thick section of the soap article over a printed matter having a bold-faced type of 14-point size. If the print can easily be read, then the bar is considered to be transparent. In the preferred embodiment, transparency or optical clarity is achieved independent of color of the bar.

### I. Preparation of the Transparent Base

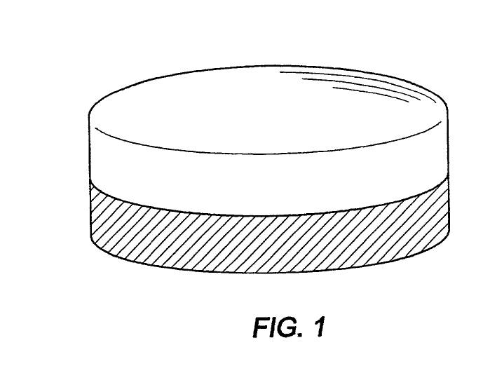

Referring now to FIG. 1, the finished bar consists dual-layers of glycerin 10 and syndet 20. The preferred embodiment of the invention utilizes a clear transparent soap base 10 of the following approximate composition. All parts, percentages and proportions referred to herein and in the appended claims are by weight unless otherwise indicated:

| Step | Ingredient | Wt. % |
|---|---|---|
| A. | Water | 8.25 |
| | Sodium Lauryl ether Sulfate | 12.25 |
| | Propylene Glycol | 20.00 |
| | Sorbitol (70% in water) | 15.00 |
| | Glycerin | 13.00 |
| B. | Stearic Acid | 13.00 |
| | Myristic Acid | 6.00 |
| C. | NaOH (50% in water) | 6.00 |
| D. | Sodium Cocoyl Isethionate | 5.00 |
| | TEA (Triethanolamine) | 1.50 |

The transparent base is preferable prepared in a 1000 pound batch by adding the ingredients in Step A into a mixing vessel and heating the batch to a temperature of approximately 140° F. The ingredients in Step B are then mixed into the batch as heat is applied. When the temperature reaches 140° F., the NaOH solution is added to the batch in a slow steady stream, which will cause the temperature to rise approximately 10 to 15° F. The batch is mixed, and heat is added if necessary to achieve a temperature of 160 to 170° F. until all of the soap pieces are broken down. The remaining ingredients are then added, and the batch is mixed until uniform.

A transparent soap color is preferably obtained by the addition of a 1% solution of any Food & Drug or Food, Drug, and cosmetic color including FD&C Red #40, Red #4, Yellow #5, D&C Red #33, in 50% propylene glycol and deionized water. A fragrance or glitter may also be added.

### II. Preparation of the Synthetic Detergent (Syndet) Base

The preferred embodiment of the invention utilizes a syndet soap base 20 of the following approximate composition. All parts, percentages and proportions referred to herein and in the appended claims are by weight unless otherwise indicated:

| Step | Ingredient | Wt. % |
|---|---|---|
| A. | Propylene Glycol | 16.0 |
| | Water | 10.0 |
| | Sodium Isethionate | 3.0 |
| | Stearic Acid | 28.0 |
| B. | Sodium Cocoyl Isethionate | 42.0 |
| C. | Titanium Dioxide | 1.0 |

The syndet base is preferable prepared in a 1000 pound batch by slowly adding the ingredients in Step A into a mixing vessel while slowly agitating the batch and heating the batch to a temperature of approximately 200° F. When the batch temperature reaches approximately 200° F., the agitation of the batch is increased as the Sodium Cocoyl Isethionate is added. The batch is continued to be mixed until all of the Sodium Cocoyl Isothionate is added and the batch is uniform. The Titanium Dioxide is then added as a preferred coloring agent. Alternatively, other coloring agents may be used. The batch is then cooled to a preferred fill temperature of 170° F.

The water should preferably be in the range of 8-12% by weight of the synthetic detergent base. A minimum of 8% is desirable to provide an effective bond to the glycerin layer. The composition should not exceed 12% in order to prevent the synthetic detergent base from becoming too soft.

### III. Pouring

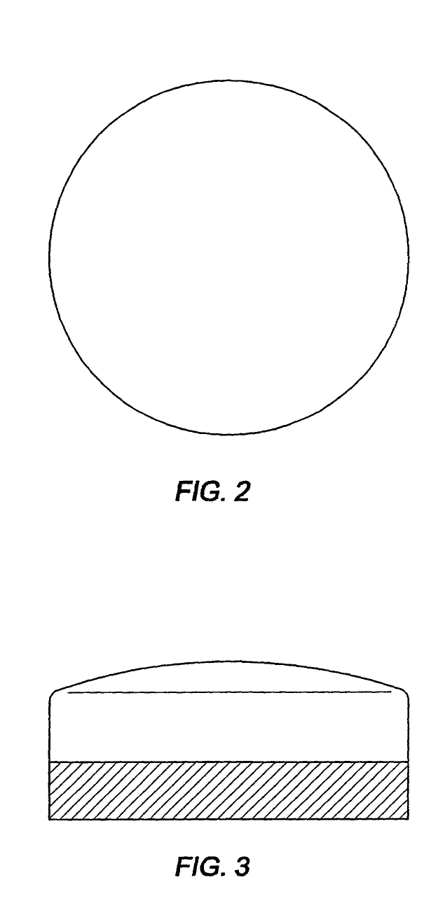

Referring now to FIG. 2, the dual-layer decoration is formed by pouring the glycerin layer 10 into a preferably circular mold having a preferable diameter of approximately 2.75 inches or 7 cm. and a depth of about 0.75 inch or 3 centimeters. After cooling to a temperature of approximately 77 to 82° F., the syndet 20 layer is poured to fill the mold. The syndet layer also has a diameter of about 2.75 inches, and is about 0.5 inches or 1 cm. thick. Once cooled to a solid state, the soap is removed. The final article preferably consists of a colored transparent top layer and a white syndet bottom layer.

It is generally desirable to use diluted dye mixtures to minimize excessive color bleed and transfer of color from the soap bar to the hands of the user, and to create a light colored pattern in the finished article.

The preferred method of manufacture of the decorative article is to pour a molten transparent soap mixture 10 into a silicon mold so that the transparent soap mixture is the top layer of the decorative soap article. Typically, a six-cavity silicone release mold will be used for a standard size bar of soap. These release molds are commercially available. Each cavity is in the shape of the finished article of soap.

The manufacture is typically performed on a conveyor production line. As the partially filled molds move down the line, cooling takes place and the bars begin to solidify. The cooling is typically enhanced in a coolant chamber maintained at approximately 34° F. The partially filled mold is cooled until the soap composition reaches a temperature of approximately 77 to 82° F., which typically requires approximately 30 minutes of cooling. The final filled mold is typically passed through the same coolant chamber to cool the final article for packaging.

The full soap cavity molds are cooled on the production line, removed from the mold and wrapped or packaged for sale. The preferable composition for manufacture is a clear transparent soap composition.

The effect of this manufacturing process is to produce a soap article with a dual-layer pattern of glycerin 10 and syndet 20. Optionally, a relatively small amount of glitter may be added to the formulation for additional decorative effect.

Although the preferred embodiment specifies a circle of 2.75 inches diameter, the shape of the finished soap bar is not limited to a circle. Rectangle, and ovals, circles, polygons, and other shapes may be used.

In alternative embodiments, the finished article may be prepared directly in blister packages or clamshell packages rather than silicone molds.

Various modifications of the formula and process will be apparent to one skilled in the art.

What is claimed is:

1. A dual layer toilet bar comprising:
a first transparent glycerin soap layer, such that the glycerin layer is transparent; and
a second opaque syndet soap layer.

2. The toilet bar of claim 1 wherein
a glitter is included in the first transparent glycerin soap layer.

3. The toilet bar of claim 1 wherein
a fragrance is included in the first transparent glycerin soap layer.

4. The toilet bar of claim 1 wherein
The first transparent glycerin soap layer is colored.

5. The toilet bar of claim 1 wherein
the toilet bar is rectangular in shape.

6. The toilet bar of claim 5 wherein
the upper surface of the toilet bar is convex.

7. A dual layer toilet bar comprising:
a first transparent glycerin soap layer, such that the glycerin layer is transparent; and
a second opaque syndet soap layer, such that the syndet layer is comprised of 14-18% by weight propylene glycol; 8-12% by weight water; 2-4% by weight sodium isethionate; 26-30% by weight stearic acid; 40-44% by weight sodium cocoyl isethionate; and 0.5-1% by weight titanium dioxide.

8. The toilet bar of claim 7 wherein
the first transparent glycerin soap layer is comprised of 18-22% by weight propylene glycol; 13-17% by weight of 70% Sorbitol in water; 11-13% by weight sodium lauryl ether sulfate; 11-15% by weight glycerin, 11-15% by weight stearic acid; 5-7% by weight myristic acid; 7-9% by weight water; 5-7% by weight of a 50% sodium hydroxide and water solution, 1-2% by weight triethanolamine; and 4-6% by weight sodium cocoyl isethionate.

9. A dual layer toilet bar comprising:
a first transparent layer, the first layer comprised of 20% by weight propylene glycol; 15% by weight of 70% Sorbitol in water; 12.25% by weight sodium lauryl ether sulfate; 13% by weight glycerin, 13% by weight stearic acid; 6% by weight myristic acid; 8.25% by weight water; 6% by weight of a 50% sodium hydroxide and water solution, 1.5% by weight triethanolamine; and 5% by weight sodium cocoyl isethionate; and a second opaque layer, the second layer comprised of 16% by weight propylene glycol; 10% by weight water; 3% by weight sodium isothionate; 28% by weight stearic acid; 42% by weight sodium cocoyl isothionate; and 1% titanium dioxide.

10. The toilet bar of claim 9 wherein
a coloring agent is added to the first transparent layer.

11. The toilet bar of claim 9 wherein
the bar is fabricated by pouring the first transparent layer into a mold and pouring the second opaque layer into the mold on top of the first transparent layer, thereby forming a dual-phase bar.

12. A method of fabricating a soap article comprised of a glycerin based-soap layer and synthetic detergent layer, the method comprising:
preparing a glycerin based soap composition;
preparing a synthetic detergent soap composition comprising 14-18% by weight propylene glycol, 8-12% by weight water, 2-4% by weight sodium isethionate, 26-30% by weight stearic acid, 40-44% by weight sodium cocoyl isethionate, and a coloring agent;
pouring the glycerin based soap composition into a mold to a height of 30-70% of the mold depth;
cooling the glycerin based soap composition in the mold to a temperature of 77-82 degrees Fahrenheit;
pouring the synthetic detergent soap composition into the mold on top of the glycerin based soap composition;
cooling the contents of the mold; and
removing the bar.
